(19) United States
(12) Patent Application Publication
(10) Pub. No.: US 2010/0004480 A1
(43) Pub. Date: Jan. 7, 2010

(54) METHODS AND COMPOSITIONS FOR INHIBITING BIOFILMS

(75) Inventors: Gary R. Eldridge, St. Louis, MO (US); Jin-Feng Hu, San Diego, CA (US)

Correspondence Address: GALLOP, JOHNSON & NEUMAN, L.C., 101 S. HANLEY, SUITE 1600, ST. LOUIS, MO, 63105, US

(73) Assignee: Sequoia Sciences, Inc., St. Louis, MO (US)

(21) Appl. No.: 12/534,331

(22) Filed: Aug. 3, 2009

Related U.S. Application Data
(63) Continuation of application No. 11/081,240, filed on Mar. 16, 2005.
(60) Provisional application No. 60/587,680, filed on Jul. 14, 2004.
(60) Provisional application No. 60/609,763, filed on Sep. 14, 2004.
(60) Provisional application No. 60/610,431, filed on Sep. 16, 2004.

(51) Int. Cl. C07C 69/732; C07C 69/734

(52) U.S. Cl. 560/61

(57) ABSTRACT

The present invention discloses compounds, compositions, and methods of using such compounds and compositions to inhibit, reduce, prevent, and remove biofilms. The invention further relates to methods of inhibiting biofilms on various substrates, such as medical devices.

### CROSS-REFERENCE TO RELATED APPLICATIONS

This application claims benefit under 35 U.S.C. §119(e) to U.S. Provisional Application Ser. No. 60/587,680, filed Jul. 14, 2004, U.S. Provisional Application Ser. No. 60/609,763, filed Sep. 14, 2004, and U.S. Provisional Application Ser. No. 60/610,431, filed Sep. 16, 2004.

The work of this invention was supported in part by a grant from the U.S. National Institutes of Health. The United States Government may have certain rights to this invention.

### FIELD OF THE INVENTION

The present invention relates to the field of biofilm inhibitors. More particularly, the present invention relates to compositions, compounds, and methods for inhibiting, reducing, preventing, and removing biofilms.

### BACKGROUND OF THE INVENTION

The demand for new compositions to treat or prevent microbial infections, e.g., bacterial infections, is often publicized and is among the greatest priorities for pharmaceutical and medical device companies, health care providers, and governments alike. Such demand is continually fueled by the rising incidence of bacterial infections that show resistance to currently-available antibiotics. For example, data have shown that approximately 70% of bacteria present in hospitals are resistant to at least one of the commonly prescribed antibiotics. While a need exists for new antibiotics to combat such resistant bacterial strains, it is further desired to discover novel methods of preventing and treating bacterial infections, i.e., using compositions and methods other than conventional antibiotics alone. Preferably, such compositions and methods would operate through a mode of action different than that of traditional antibiotics.

Biofilms represent a target of new compositions for inhibiting, reducing, preventing, and removing microbial infections. A biofilm is a conglomerate of microorganisms, such as bacteria, embedded in a hydrated matrix of exopolymers, typically polysaccharides, and other macromolecules. Biofilms generally protect, e.g., bacteria from antibiotics and immune systems. In fact, it is believed that biofilms are partly responsible for increasing the rates of antibiotic resistance. More particularly, it has been found that biofilms hinder the ability of antibiotics to access such bacteria to completely eradicate their existence.

In light of the foregoing, there is a demand for compositions and methods for inhibiting, reducing, preventing, and removing biofilms. Such compositions and methods, preferably, (i) act directly on the biological mechanism that protects microorganisms from antibiotics and (ii), generally, decrease the rate at which icroorganisms may acquire resistance to antibiotics.

### SUMMARY OF THE INVENTION

One embodiment of the present invention is a method for inhibiting, reducing, preventing, and removing biofilms. This method comprises providing to a system in need thereof an effective amount of a compound selected from the group consisting of:

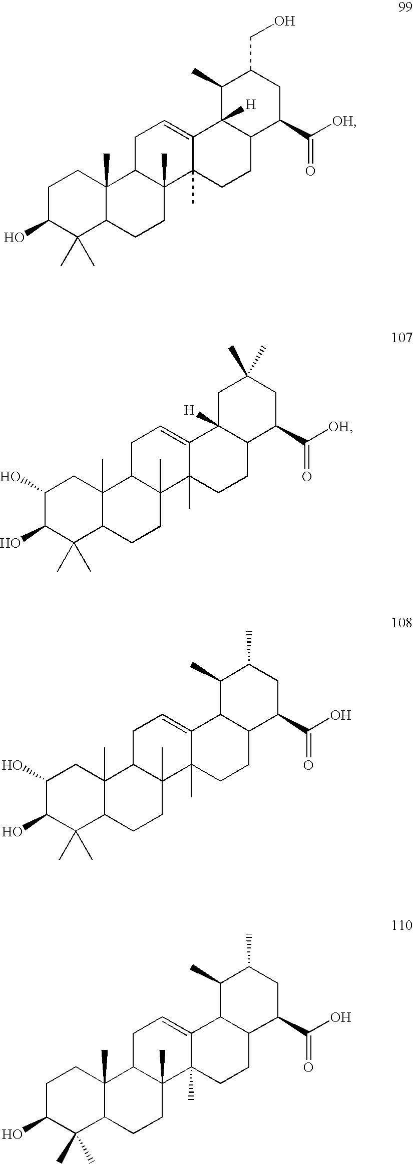

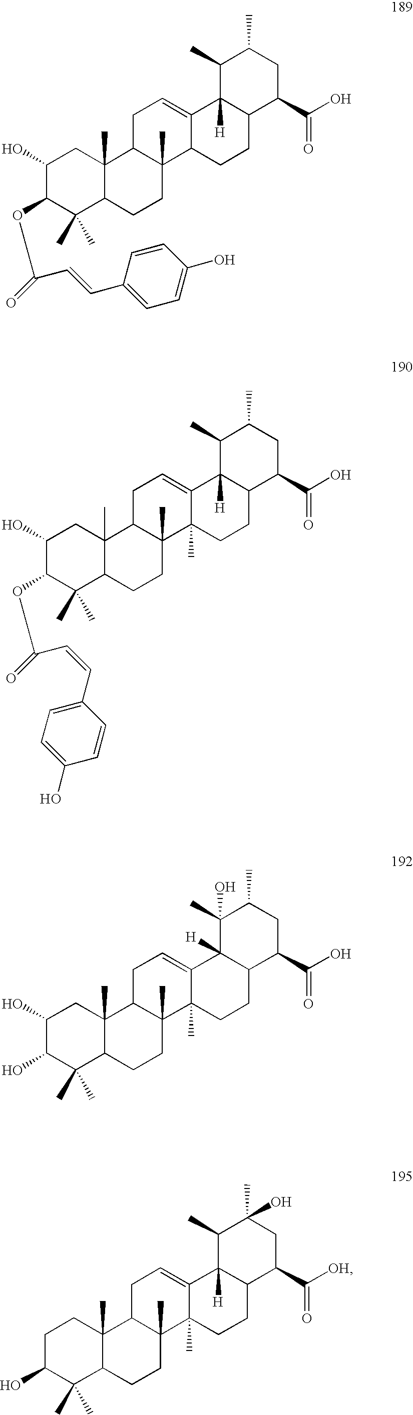

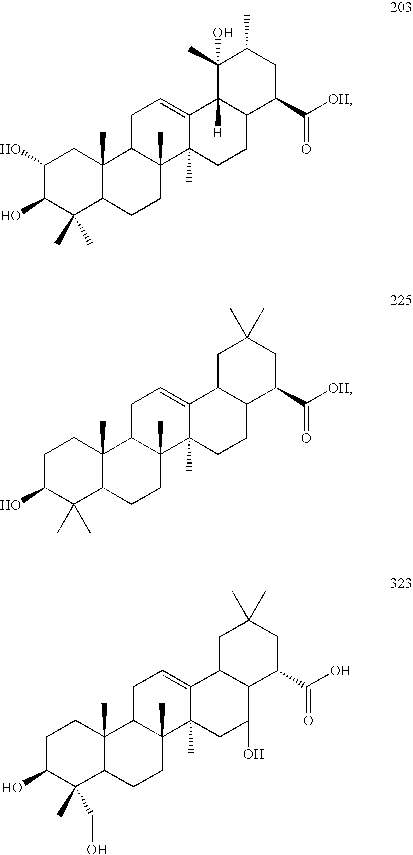

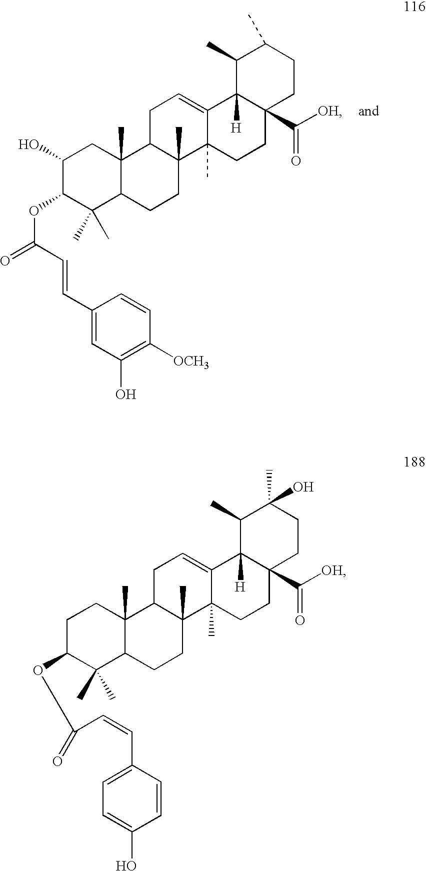

including salts, hydrates, solvates, N-oxides, and combinations thereof.

In another embodiment, the invention provides methods for inhibiting, reducing, preventing, and removing biofilms in or on a medical device, which comprises providing a medical device in need thereof with an effective amount of one or more of the compounds identified above. Non-limiting examples of medical devices that may be used in such embodiments include central venous catheters, urinary catheters, endotracheal tubes, mechanical heart valves, pacemakers, vascular grafts, stents, and prosthetic joints. Still further, in certain embodiments, the invention provides methods for inhibiting, reducing, preventing, and removing biofilms in or on transportation vehicles, plants, and other substrates.

Another embodiment of the present invention includes compounds according to the chemical structures:

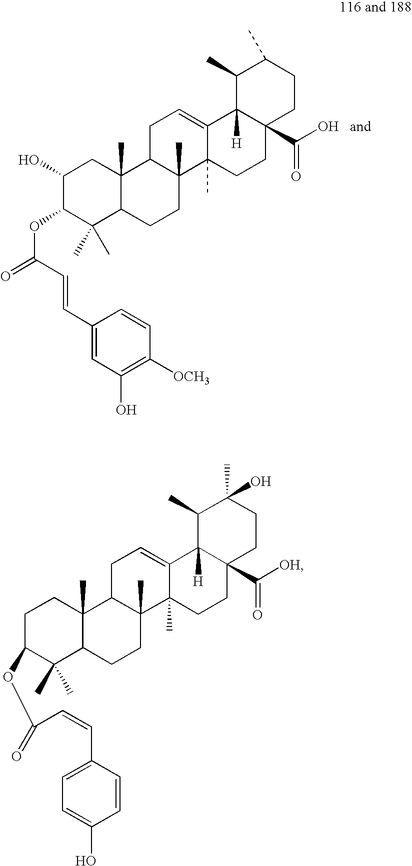

including salts, hydrates, solvates, N-oxides, and mixtures thereof.

### BRIEF DESCRIPTION OF THE FIGURES

FIG. 1 is a graph illustrating that compound 108 does not inhibit the growth of E. coli in LB media from 1 to 15 μg/mL.

FIG. 2 is a graph illustrating the dose/response behavior of compound 108 at 0 and 15 μg/mL against E. coli in LB media when added to the system described below along with the inoculum.

FIG. 3 is a graph illustrating the dose/response behavior of compound 108 at 0, 1, 2, 5, and 10 μg/mL against E. coli in LB media when added to the system described below along with the inoculum.

FIG. 4 is a graph illustrating the dose/response behavior of compound 108 at 0, 1, 2, 5, and 10 μg/mL against E. coli in LB media when added to the system described below 24 hours after inoculation.

FIG. 5 is a graph illustrating the dose/response behavior of compound 108 at 0, 1, 2, 5, and 10 μg/mL against E. coli in LB media (with 0.2% glucose) when added to the system described below along with the inoculum.

### DETAILED DESCRIPTION OF THE INVENTION

As used herein, the term “biofilm” and the like means an extracellular matrix in which microorganisms are dispersed and/or form colonies. The biofilm typically is made of polysaccharides and other macromolecules. In addition, in the present invention, the phrase “inhibiting a biofilm,” and like phrases, means the prevention of biofilm growth, reduction in the rate of biofilm growth, partial eradication of existing biofilm, and/or complete eradication of existing biofilm.

In one embodiment of the present invention, methods for inhibiting, reducing, preventing, and removing biofilms are provided, which comprise providing to a system in need thereof one or more of the following compounds:

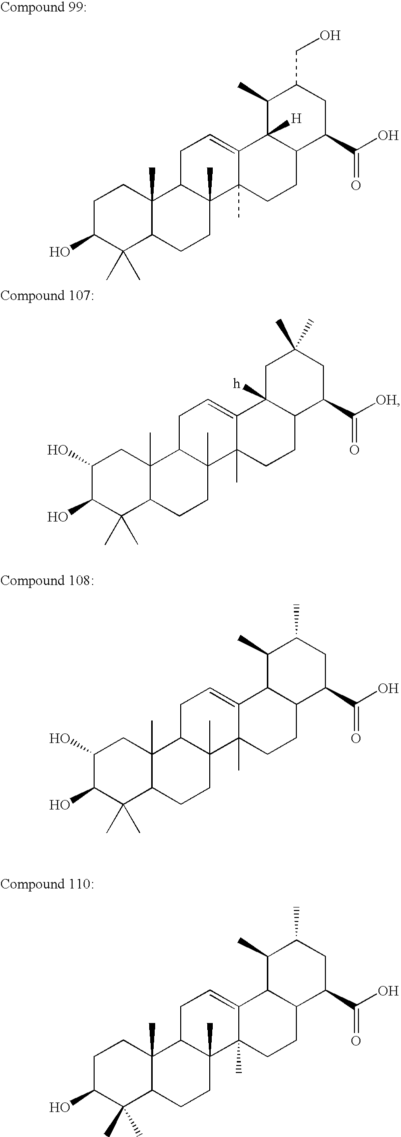

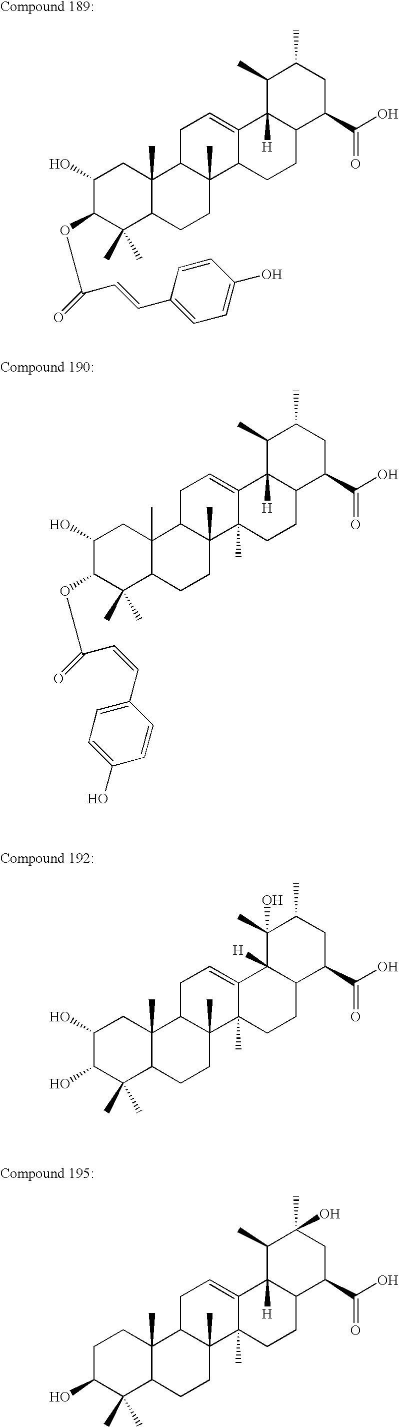

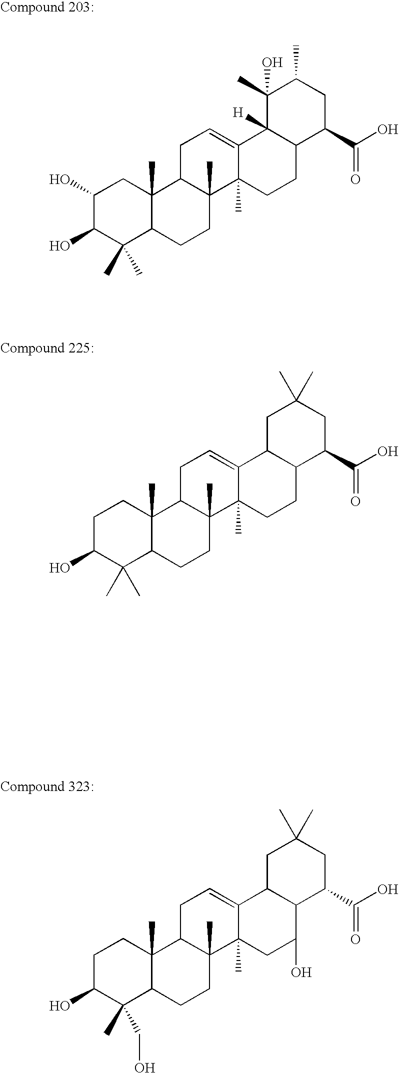

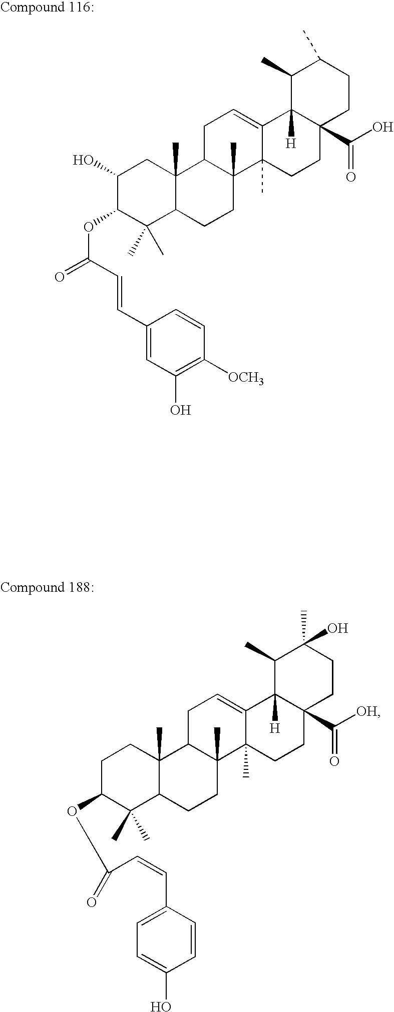

including salts, hydrates, solvates, N-oxides, and mixtures thereof.

In such methods, an antibiotic or biocide may be administered with the compound or administered separately. In the present invention, any conventional biocide may be used. Representative examples of biocides that may be used in the present invention include isothiazolone, derivatives thereof, compounds having isothiazolone functions, 3-isothiazolone, 5-chloro-2-methyl-3-isothiazolone, 1-methyl-3,5,7-triaza-1-azoniatricyclo (3.3.1.1) deoane chloride, 4,5-dichloro-2-octyl-3-isothiazolone, 2-bromo-2-nitropropanediol, 5-bromo-5-nitro dioxane, thiocyanomethylthiobenzothiazole, 4,5-dichloro-2-octyl-3-isothiazolone and 2n-octyl-3-isothiazolone, tetrachloroisophalonitrile, 1,2-benzisothiazolin-3-one, 2-methyl-4,5-trimethylene-4-isothiazolin-3-one, 5-chloro-2-methyl-4-isothiazolin-3-one, 2-methyl-4-isothiazolin-3-one, 4-(2-nitrobutyl)morpholine, beta-nitrostyrene (“NS”), beta-bromo-beta-nitrostyrene (“BNS”), methylchloro/isothiazolone (“IZN”), methylenebisthiocyanate (“MBT”), 2,2-dibromo-3-nitrilopropionamide (“DBNPA”), 2-bromo-2-bromomethyl-glutaronitrile (“BBMGN”), alkyldimethylbenzylammoniun chloride (“ADBAC”), and beta-nitrovinyl furan (“NVF”), 2-methyl-3-isothiazolone, methylene bisthiocyanate, p-tolyldiiodotmethyl sulfone, 2-methylthio-4-tertbutylamino-6-cyclopropylamino-s-triazine, N,N-dimethyl-N′phenyl-(N′fluorodichloromethylthio)sulfamide, antibiotics, sulfamides, tetracycline, isothiazolone derivatives, N-(cyclo)alkyl-isothiazolone, benzisothiazolin-3-one, and mixtures of the foregoing.

Other examples of biocides that may be combined with one or more of the biocides (and/or biofilm-inhibiting compounds) listed above include bicyclic oxazolidoines and their mixtures, amine-based bactericide, polyacrolein copolymer, 4,4-dipethyloxazolidine, 2((hydroxymethyl)-amino) ethanol, mixtures of 1,2-benzisothiazolone-3-one with one or more amines, tetrahydro-3,5-dimethyl-2H-1,3,5-thiadiazine-2-thione, 1,2-benzisothiazolin-3-one, tetrachloroisophthalonitrile, N-cyclopropyl-N-(1,1-dimethylethyl)-6-(methylthio)-1,3,5-triazine-2,4-diamine, mixtures of N-cyclopropyl-N-(1,1-dimethylethyl)-6-(methylthio)-1,3,5-triazine-2,4-diamine with tetrachloroisophthalonitrile, mixtures of tetrachloroisophthalonitrile with 3-iodo-2-propynylbutyl carbamate, N-(trichloromethylthio)-phthalimide, 3-iodo-2-propynylbutyl carbamate, tetrachloroisophthalonitrile, and mixtures of the foregoing.

Non-limiting examples of antibiotics that may be used in connection with the present invention include amoxicillin, penicillin, clarithromycin, cefaclor, cefuroxime, cefprozil, ciprofloxacin, clindamycin, fluconazole, dicloxacillin, erythromycin, metronidazole, ofloxacin, griseofulvin, sulfisoxazole, griseofulvin, cephalexin, terbinafine, levofloxacin, loracarbef, nitrofurantoin, minocycline, clotrimazole, nystatin, ketoconazole, cefdinir, ampicillin, trimethoprimsulfamethoxazole, itraconazole, cefixime, mebendazole, doxycycline, sparfloxacin, azithromycin, and mixtures of the foregoing.

The biofilm-inhibiting compositions of the present invention may further be administered in connection with pharmaceutically acceptable carriers. Examples of such carriers include carriers for solid preparations, such as lactose, sucrose, glucose, starch and crystalline cellulose; binders such as starch, hydroxypropylcellulose and carboxymethylcellulose; lubricants such as talc, stearic acid and stearate salts; and carriers for liquid preparations, such as sucrose, glucose, fructose, invert sugar, sorbitol, xylitol, glycerin, gum arabic, tragacanth and carboxymethylcellulose sodium.

The biofilm-inhibiting compounds may also be coupled with soluble polymers as targetable drug carriers. Such polymers may include polyvinylpyrrolidone, pyran copolymer, polyhydroxypropylmethacrylamide phenyl, polyhydroxyethylaspartamide-phenol, or polyethyleneoxide-polylysine substituted with palmitoyl residues. Furthermore, the biofilm-inhibiting compounds may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and cross-linked or amphipathic block copolymers of hydrogels.

In the present invention, the biofilm-inhibiting compounds described herein may be provided to a system before, during, or after a biofilm has formed. Thus, such compounds may be administered after a system has developed a biofilm or as a prophylactic to prevent the formation (or reformation) of a biofilm.

In certain preferred embodiments, one or more of the biofilm-inhibiting compounds may be applied to the surface of a substrate. The substrate may be made from any material to which such compound (or a composition containing the compound) may be applied. Representative examples of the kinds of materials from which the substrate may be made include porous materials, soft materials, hard materials, semi-hard materials, regenerating materials, and non-regenerating materials. Preferably, the substrate is made from an inert material selected from the group consisting of a polymer, a metal, an alloy, and combinations thereof.

Preferably, the substrate is a surface of a device that is susceptible to biofilm formation. Examples of suitable substrate surfaces according to the present invention include vessel hulls, automobile surfaces, air plane surfaces, membranes, filters, and industrial equipment.

The substrate may also include medical devices, instruments, and implants. Examples of such medical devices, instruments, and implants include any object that is capable of being implanted temporarily or permanently into a mammalian organism, such as a human. Representative medical devices, instruments, and implants that may be used according to the present invention include, for example, central venous catheters, urinary catheters, endotracheal tubes, mechanical heart valves, pacemakers, vascular grafts, stents, and prosthetic joints.

In additional embodiments of the present invention, the biofilm-inhibiting compounds (or compositions containing such compounds) may be administered to a plant, such as a surface of a plant (including commercial crop varieties) to prevent or inhibit the formation of a biofilm on the plant and, preferably, prevent or reduce biofilm growth and bacterial colonization that may harm such plants, decrease yield, etc. Representative types of plants to which the compounds or compositions of the present invention may be applied include, for example, corn, maize, soybean, wheat, rice, and canola plants.

In still further embodiments of the present invention, certain novel compounds (and compositions containing such compounds) are provided having the chemical structures shown below:

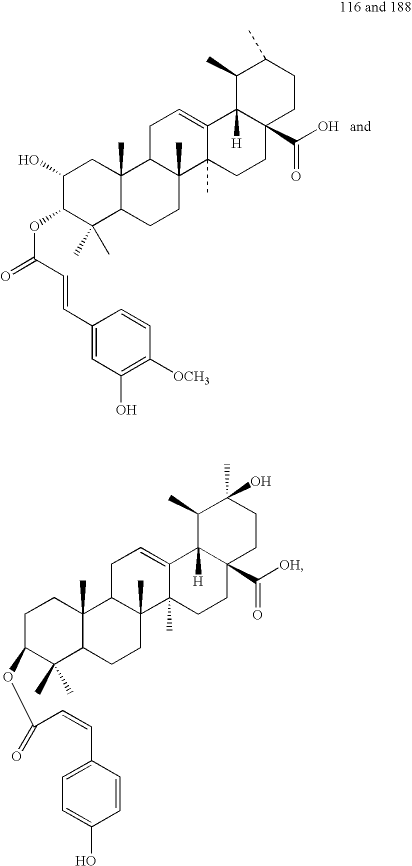

including salts, hydrates, solvates, N-oxides, and mixtures thereof. The present invention provides that such compounds are useful in the methods described herein, including methods of inhibiting, preventing, reducing, and treating biofilms on or in a substrate, such as medical devices, transportation vehicles, plants, and others.

Compound 116, 3α-O-trans-feruloyl-2α-hydroxy-12-ursen-28-oic acid, has a chemical formula of C₄₀H₅₆O₇ and a molecular weight of approximately 648.87. Compound 116 may be extracted and purified from Diospyros dendo (Gabon, Africa), as described further below. Compound 188, 3β-O-cis-p-coumaroyl-20β-hydroxy-12-ursen-28-oic acid, has a chemical formula of C₃₉H₅₄O₆ and a molecular weight of approximately 618.84. Compound 188 may also be extracted and purified from Diospyros dendo. Compounds 116 and 188 are soluble in methanol, ethanol, and dimethyl sufoxide (“DMSO”) from about 4 to 8 mg/mL.

The compounds described herein to be useful in practicing the invention contain one or more asymmetric centers and thus give rise to enantiomers, diastereomers, and other stereoisomeric forms that may be defined, in terms of absolute stereochemistry, as (R)— or (S)—. The present invention encompasses all such possible isomers, as well as their racemic and optically pure forms. Optical isomers may be prepared from their respective optically active precursors, or by resolving the racemic mixtures. Such resolution may be carried out in the presence of a resolving agent, by chromatography, or by repeated crystallization or by some combination of such techniques which are known to those skilled in the art.

When the compounds described herein contain olefinic double bonds, other unsaturation, or other centers of geometric asymmetry, and unless specified otherwise, it is intended that the compounds include both E and Z geometric isomers or cis- and trans-isomers. Similarly, all tautomeric forms are intended to be encompassed by the present invention. The configuration of any carbon-carbon double bond appearing herein is selected for convenience only and is not intended to designate a particular configuration unless the text so states; thus, a carbon-carbon double bond or carbon-heteroatom double bond depicted arbitrarily herein as trans may be cis, trans, or a mixture of the two in any proportion.

Many of the compounds described herein may be isolated and purified from plant material. Table 1 shows the representative plant species from which various compounds described herein have been isolated and purified.

TABLE 1

| Compound | Plant Species |
|---|---|
| 99 | Arctostaphylos tomentosa (California, USA); |
| | Arctostaphylos edmundsii (California, USA); and |
| | Phyla nodiflora (Texas, USA) |
| 107 | Diospyros dendo (Gabon, Africa) |
| 108 | Diospyros dendo (Gabon, Africa) |
| 110 | Diospyros dendo (Gabon, Africa) |
| | Arctostaphylos tomentosa (California, USA); |
| | Arctostaphylos edmundsii (California, USA); and |
| | Malus domestica (California, USA) |
| 189 | Diospyros dendo (Gabon, Africa); and |
| | Malus domestica (California, USA) |
| 190 | Diospyros dendo (Gabon, Africa) |
| 192 | Brazzeia soyauxii (Gabon, Africa) |
| 195 | Arctostaphylos tomentosa (California, USA); and |
| | Arctostaphylos edmundsii (California, USA); |
| 203 | Brazzeia soyauxii (Gabon, Africa) |
| 116 | Diospyros dendo (Gabon, Africa) |
| 188 | Diospyros dendo (Gabon, Africa) |

Those of ordinary skill in the art will appreciate that the compounds listed in Table 1 may be found in and isolated from other varieties within the respective plant family, genus, etc. represented therein. In addition, purified forms of compounds 225 and 323 are commercially-available. For example, compound 225 may be purchased from Sigma-Aldrich Chemical Company (St. Louis, Mo., USA) and compound 323, may be purchased from Chromadex (Santa Ana, Calif., USA).

The compounds disclosed herein may be separated and purified from the plant sources described above using methods such as column chromatography, high pressure liquid cliromatography (HPLC), and/or recrystallization. As will be appreciated by the skilled artisan, further methods of synthetically producing and derivatizing the compounds disclosed herein will be evident from this specification. Additionally, the various isolation, purification, and/or synthetic steps may be performed in an alternate sequence or order to produce the desired compounds.

For purposes of illustration, the following provides a non-limiting example of a general procedure that may be employed to isolate and purify the compounds described herein. First, an extraction step may be carried out by grinding dried plant material to a homogenous powder and sonicating the powder in an organic solvent, such as a mixture of Ethanol:Ethyl Acetate (EtOH:EtOAc) (50:50), and shaking the resulting mixture vigorously for exhaustive extractions. Next, flash chromatographic separation may be carried out by dissolving the organic extract in 5 mL of a solvent, such as Methanol:Ethyl Acetate (MeOH:EtOAc) (50:50), adsorbing it onto silica powder and bringing the dried powder onto a silica column and eluting on the flash chromatography system using a step gradient of (1) 75% hexanes, 25% EtOAc, (2) 50% hexanes, 50% EtOAc, (3) 100% EtOAc, (4) 75% EtOAc, 25% MeOH, and (5) 50% EtOAc, 50% MeOH. The flash fraction containing highly lipophilic material may be discarded, whereas the remaining fractions may be dried, such as by rotary evaporation. One or more flash fractions may be screened for the presence of tannins using liquid chromatography/mass spectrometry (LC-MS) (and passed over a polyamide column if results are positive). See Anderson, K. J.; Teuber, S. S.; Gobeille, A.; Cremin, P. A.; Waterhouse, A. L.; Steinberg, F. M. J. Nutr. 2001, 131, 2837-2842.

Preparative HPLC separation may then be carried out. The flash fraction material may be dissolved into either MeOH:EtOAc (70:30) or 100% MeOH (and filtered when necessary). The fractions may be further separated into several individual fractions, such as 40, using a device such as a parallel four-channel preparative HPLC system. A different gradient may be applied to each flash fraction for adequate separation. For example, a first fraction may be eluted in 40-80% acetonitrile in water; a second in 30-70% acetonitrile in water; a third in 20-60% acetonitrile in water; a fourth in 10-50% acetonitrile in water; and so on. Of course, those of ordinary skill in the art will appreciate that other organic solvents, and combinations, gradients, and ratios thereof, may be used during HPLC separation—which may depend on the nature of the plant extracts, extraction procedures employed, desired compound and others.

The resulting HPLC fractions may be dried in an evaporator. The HPLC fractions may then be transferred to plates, such as 96-deep-well plates, using a liquid handling system (e.g., Packard MultiProbe II). Next, the molecular weights of the materials in the samples may be determined using a parallel eight-channel liquid chromatography electrospray detection mass spectrometry (LC-ELSD-MS) system with chromatographic conditions of 5% acetonitrile in water for the first minute, a linear gradient of acetonitrile from 5% to 95% in eight minutes, followed by 95% acetonitrile in water for a minute. Under such chromatographic conditions, the column is equilibrated at 5% acetonitrile in water after each analysis.

Data processing for determining the appropriate dilution for each sample for normalization may be automated, for example, with computer software to extract all graphic information, such as retention times, mass spectra, and peak integrations, and to convert such information to text to allow it to be transferred to a database for storage and analysis. An example of such computer software is MicroMass MassLynx, offered by Matrix Science, Inc. (Boston, Mass.). In addition, the invention provides that data analysis may, optionally, be performed using OpenLynx Software offered by Waters Corporation (Milford, Mass.) and Extractor—a customized software package developed for Sequoia Sciences, Inc. by Koch Associates (La Jolla, Calif.). Still further, the structure of the desired compound may optionally be confirmed using nuclear magnetic resonance spectroscopy (NMR), as described further below.

In addition to isolating such compounds from the plant material described herein, the compounds may, alternatively, be prepared semi-synthetically. If prepared semi-synthetically, a typical starting material may be, for example, compound 110, 225, or any other compound disclosed herein. The plurality of compounds useful in the present invention and described herein may be produced, for example, by first extracting and purifying a sample compound, such as compound 110 or 225, and subsequently derivatizing the starting compound to remove and/or append certain desired functional groups to such compound.

Synthetic chemistry transformations and protecting group methodologies (protection and deprotection) useful in synthesizing and/or derivatizing the compounds described herein are known in the art and include, for example, those described in R. Larock, Comprehensive Organic Transformations, VCH Publishers (1989); T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

The compounds disclosed herein may be modified by appending any desired functionalities to enhance selective biological properties. Such modifications are known in the art and may include those which increase biological penetration into a given system or substrate.

The following examples are provided to illustrate further aspects of the present invention. These examples are illustrative only and are not intended to limit the scope of the invention in any way.

### Examples

### Example 1

### Inhibition of Biofilms

A microtiter plate assay was used to quantitatively measure the effect each tested compound had on the ability of bacteria to form a biofilm. In this example, a concentrated solution of each compound tested was loaded separately into three separate wells of a polystyrene microtiter plate. In addition, each assay included triplicate wells correlating to negative and positive controls. For the positive controls, biofilm inhibitors of known activity were used, whereas no inhibitors were added to wells correlating to negative controls.

Next, 150 μl of sterile media was added to each well (LB media with 0.2% glucose)—followed by 50 μl of the appropriate bacterial inoculum. Thus, each well contained a final volume of approximately 200 μl (not including the volume of the concentrated inhibitor). The final concentration of each biofilm inhibitor tested in the assay was 10 μg/ml. The microtiter plates were then placed on a shaker for 24 hours at room temperature.

After the 24 hour incubation period, the microtiter plate was removed from the shaker, rinsed, and stained. During the rinsing step, the test compound, media, and bacterial inoculum solution was drained from the plate, approximately 300 μl of 0.1 M phosphate buffered saline (PBS) was added to each well, which was subsequently drained from the plate. The rinsing step removed any suspended cells from the assay. 0.1% crystal violet stain was added to each well for approximately 20 minutes.

Next, the crystal violet solution was drained from the microtiter plate. The plate was rinsed with PBS as described above four (4) times to remove any excess stain from the plate. Following the PBS rinsing steps, the plate was eluted with 250 μl/well of ethanol, which improved the detection of the stain. The plate was immediately analyzed spectrophotometrically at 540 nm using a microtiter plate reader. The inhibitory effect each compound had on the bacteria's ability to form a biofilm on the surface of each well was determined as follows:

The absorbance values observed for each set of three (3) wells correlating with a test compound or control were averaged. The average absorbance value for each test compound was compared to the average absorbance value of the negative control (the positive control was employed to verify proper assay function). In general, biofilm inhibition activity is inversely proportional to absorbance values, whereby, for example, low absorbance values correlate with significant inhibition activity and high absorbance values correlate with small or no inhibition activity. The approximate percent inhibition observed for each compound was calculated by comparing the average absorbance value for each test compound to the average absorbance value for the negative controls. Table 2 summarizes the average percent inhibition observed for the tested compounds listed against biofilms generated by Pseudomonas aeruginosa.

TABLE 2

| | Biofilm Inhibition- |
|---|---|
| Compound | Pseudomonas aeruginosa |
| 99 | 30% |
| 107 | 46% |
| 108 | 52% |
| 110 | 35% |
| 116 | 48% |
| 188 | 62% |
| 189 | 35% |
| 192 | 32% |
| 195 | 25% |
| 203 | 43% |
| 225 | 35% |

As shown in Table 2, the biofilm inhibitors referenced therein exhibited significant biofilm inhibition activity. Notably, in wells correlating to compounds 188, 108, and 116, a reduction in biofilm mass of 62%, 52%, and 48% respectively, was observed.

To further demonstrate the ability of the compounds disclosed herein to inhibit biofilm formation generated by a diverse array of microorganisms, compounds 108, 110, 225, and 323 were evaluated for their activity against Escherichia coli. Such compounds were evaluated using an assay adapted from a protocol reported in Pratt and Kolter, 1998, Molecular Microbiology, 30: 285-293; and Li et al., 2001, J. Bacteriol., 183: 897-908. More particularly, E. coli JM109 was grown in LB with or without 0.2% glucose in 96 well plates at 37° C. for two days without shaking. The test compounds were added to separate wells containing the inoculate and tested in 3-4 replicates at a final concentration of 10 μg/ml. Negative controls included only ethanol (the solvent for each test compound).

To quantify the biofilm mass, the suspension culture was removed, the biofilm was washed three times with water, and then stained with 0.1% crystal violet for 20 minutes. Next, the plates were washed three times with water and analyzed by spectrophotometry at 540 μm to quantify the biofilm mass (by comparing the biofilm mass in the wells containing a test compound to that of the control wells). The data are summarized in Table 3 below.

TABLE 3

| | Biofilm Inhibition- |
|---|---|
| Compounds | Escherichia coli |
| 108 | 74% |
| 110 | 80% |
| 225 | 35% |
| 323 | 35% |

As shown in Table 3, the biofilm inhibitors referenced therein exhibited significant biofilm inhibition activity against E. coli. Notably, in wells correlating to compounds 110 and 108, a reduction in biofilm mass of 80% and 74%, respectively, was observed. Compound 110 was further tested against Staphylococcus epidermidis. Using the assay described above, at the final concentration of 10 μg/ml, compound 110 was shown to inhibit biofilm formation by S. epidermidis by approximately 25%. The foregoing data show that the biofilm inhibitors described herein are capable of significantly reducing biofilm growth produced by a wide variety of bacteria.

### Example 2

### Evaluation of Biofilm Inhibition Using a Flow Apparatus

To validate the data shown in the previous example, the biofilm inhibition activity of compound 116 was measured using a microscope flow cell system—which is considered to be an extremely durable and precise method among those skilled in the art. In general, flow cell analysis begins by inoculating a microorganism into test and control flow cells along with the appropriate growth media (which may vary depending upon the organism involved). The microorganisms are maintained in the flow cells until they have produced biofilms having the desired coverage and thickness. Thereafter, the test flow cells are treated with appropriate concentrations of test inhibitors. Typically, one or more flow cells serve as negative controls, which are void of any biofilm inhibiting compositions. Such negative controls may be used as reference points when measuring biofilm inhibition activity.

After the appropriate flow cells are treated with the test inhibitors, Live/Dead stain is applied to each flow cell for approximately 45 minutes. Any currently-available Live/Dead stain may be employed in this procedure, such as LIVE BacLight™ Bacterial Gram stain (Molecular Probes, Inc., Eugene, Oreg., USA). The biofilm is subsequently rinsed with sterile media to remove excess stain, and the sample is transferred to a confocal microscope for imaging. The Live/Dead stain differentially stains the bacterial cells with green for Live and red for Dead. In a flow cell environment, it is generally understood that bacteria require the presence of biofilms to propagate. Accordingly, the activity of a biofilm inhibitor may be indirectly measured by comparing the number of live bacterial cells included in a flow cell that contained a test inhibitor to that of a negative control.

In this example, flow cell experiments were conducted as described above, wherein compound 116 was tested at 10 μg/mL against P. aeruginosa in LB media with 0.2% glucose. Compound 116 was applied to the test flow cells approximately four (4) hours after the P. aeruginosa inoculum was added. The negative control flow cells included the inoculum in LB media with 0.2% glucose without any inhibitors. After compound 116 was added to the test flow cells, the system was allowed to incubate. The test and control cells were subsequently stained and analyzed as described above.

Table 4 below summarizes the results of the analysis of compound 116. In this example, the number of live colony forming units (CFU) detected in the sample flow cells were compared to that of the negative control to indirectly measure biofilm inhibition. As shown, an approximate biofilm reduction of 47% was observed for compound 116 against P. aeruginosa, which corroborates the data observed using the microtiter assay described in the previous example (showing a 48% reduction in biofilm against P. aeruginosa).

TABLE 4

| | Neg. Control | Flow Cell with | | |
|---|---|---|---|---|
| | Flow Cell | Compound 116 | Log | Percent |
| | (CFU) | (CFU) | Reduction | Reduction |
| | 4.26E+06 | 2.06E+06 | | |
| | 2.41E+06 | 1.46E+06 | | |
| Average | 3.33E+06 | 1.76E+06 | 0.28 | 47% |
| Std. Dev. | 1.31E+06 | 4.21E+05 | | |

### Example 3

### Evaluation of Biofilm Inhibition Using a Rotating Disk Reactor

In this example, compound 110 was tested for biofilm inhibition with Pseudomonas aeruginosa in combination with Tobramycin. Specifically, biofilm formation of P. aeruginosa was evaluated using a standardized biofilm method with a rotating disk reactor (RDR), which is also known as “ASTM Standard Method #E-2196-02.” The rotating disk reactor consisted of a one-liter glass beaker fitted with a drain spout. The bottom of the vessel contained a magnetically driven rotor with six 1.27 cm diameter coupons constructed from polystyrene. The rotor consisted of a star-head magnetic stir bar upon which a disk was affixed to hold the coupons. The vessel (with the stir bar) was placed on a stir plate and rotated to provide fluid shear. A nutrient solution (AB Trace Medium with 0.3 mM glucose, see Table 5 below for specific formulation) was added through a stopper in the top of the reactor at a flow rate of 5 mL/min.

TABLE 5

| Component | Formula | Concentration (g/L) |
|---|---|---|
| Disodium phosphate | Na₂HPO₄ | 6.0 |
| Monopotassuim phosphate | KH₂PO₄ | 3.0 |
| Sodium Chloride | NaCl | 3.0 |
| Ammonium sulfate | (NH₄)₂SO₄ | 2.0 |
| Magnesium chloride | MgCl₂ | 0.2 |
| Glucose | C₆O₁₂H₆ | 0.054 |
| Calcium chloride | CaCl₂ | 0.010 |
| Sodium sulfate | Na₂SO₄ | 0.011 |
| Ferric chloride | FeCl₃ | 0.00050 |

The reactor volume was approximately 180 mL. At a volume of 180 mL, the residence time of the reactors was 36 minutes. The reactors were operated at room temperature (c.a. 26° C.).

In this example, two RDRs were operated in parallel with one receiving compound 110 and the other serving as an untreated control. The RDRs were sterilized by autoclave, then filled with sterile medium and inoculated with Pseudomonas aeruginosa strain PAO1. The reactors were then incubated at room temperature in batch mode (no medium flow) for a period of 24 hours, after which flow was initiated for an additional 24 hour incubation. Compound 110 was dissolved in 10 ml ethanol to achieve a concentration of 1.8 mg/mL. After the 48 hours of biofilm development described above, 10 mL of the 1.8 mg/mL ethanol-compound 110 solution was added to the reactor to achieve a final concentration of approximately 100 μg/mL. Control reactors received 10 mL of ethanol. The reactors were then incubated an additional 24 hours in batch (no flow) mode.

Following the incubation period, the six coupons were removed from each reactor and placed in 12-well polystyrene tissue culture plates with wells containing either 2 mL of a 100 μg/mL tobramycin solution or 2 mL of PBS. The plates were incubated at room temperature for two hours. The coupons were then rinsed by three transfers to plates containing 2 mL of fresh PBS. For each of the RDR reactors, four sets of three coupons were obtained: a first set that was not treated with compound 110 or tobramycin, a second set treated only with tobramycin, a third set treated only with compound 110, and a fourth set treated with compound 110 and tobramycin.

After rinsing, two coupons of each set of three were placed in 10 mL of PBS and sonicated for five minutes to remove and disperse biofilm cells. The resulting bacterial suspensions were then serially diluted in PBS and plated on tryptic soy agar plates for enumeration of culturable bacteria. The plates were incubated for 24 hours at 37° C. before colony forming units (CFU) were determined. Table 6 below summarizes the results observed in this example.

TABLE 6

| Treatment | CFU (in log 10) |
|---|---|
| Tobramycin + Compound 110 | 5.9 |
| Compound 110 | 7.1 |
| Tobramycin | 6.1 |
| Control | 7.3 |

As shown in Table 6, the treatment of compound 110 in combination with tobramycin resulted in a 0.2 log₁₀ reduction of CFUs for P. aeruginosa than with tobramycin by itself (and, furthermore, resulted in a 1.4 log₁₀ reduction of CFUs for P. aeruginosa compared to the control). The results clearly demonstrate that compound 110 increased biofilm susceptibility to tobramycin by modifying the biofilm.

### Example 4

### Minimum Inhibitory Concentration (MIC) Determination

The minimum inhibitory concentrations (MICs) for several of the specific biofilm inhibitors described herein were determined against several targets, including P. aeruginosa, E coli, and Staphylococcus aureus. The MICs of the biofilm inhibitors were measured using the reference broth microdilution method recommended by the National Committee for Clinical Laboratory Standards (NCCLS).

According to the NCCLS method, a 96-well microtiter assay was used to assess the inhibition activity of the various compounds at concentrations ranging from 5 μg/mL to 128 μg/mL. In each assay, positive and negative controls were employed, wherein the positive control included an antimicrobial agent with a known potency range that was used to ensure proper assay performance. The negative control, generally, included the solvent in which the test compounds were dissolved. In this example, the wells correlating to the negative controls included ethanol (without any biofilm inhibitors).

In this example, 150 μl of sterile media was added to each well (LB media with 0.2% glucose), along with appropriate volumes of concentrated solutions of the inhibitors to be tested, followed by 50 μl of bacterial inoculum. Thus, each well contained a final volume of approximately 200 μl (not including the volume of the concentrated inhibitor). The final concentration of each biofilm inhibitor tested in the assay ranged from 5 μg/mL to 128 μg/mL. After bacterial inoculation and loading of test and control samples, the plate was incubated in an ambient air environment at 35° C. for 20-24 hours. The plate was subsequently rinsed, stained, and analyzed in accordance with the procedures described in Example 1.

Tables 7, 8, and 9 summarize the data observed and, specifically, the MICs calculated for the several compounds referenced therein against P. aeruginosa, E. coli, and S. aureus, respectively. The minimum inhibitory concentrations were calculated as the lowest inhibitor concentration at which biofilm inhibition was observed.

TABLE 7

| | MIC (μg/mL) - |
|---|---|
| Compound | Pseudomonas aeruginosa |
| 99 | >10 |
| 107 | >10 |
| 108 | >10 |
| 110 | >128 |
| 116 | >10 |
| 188 | >10 |
| 189 | >10 |
| 192 | >10 |
| 195 | >10 |
| 203 | >10 |

TABLE 8

| | MIC (μg/mL) - |
|---|---|
| Compound | Escherichia coli |
| 108 | >15 |
| 110 | >10 |
| 225 | >10 |
| 323 | >10 |

TABLE 9

| | MIC (μg/mL) - |
|---|---|
| Compound | Staphylococcus aureus |
| 99 | >32 |
| 108 | >32 |
| 195 | >8 |

### Example 4

### Selectivity of the Biofilm Inhibitors

In this example, two (2) representative compounds useful in the present invention, namely, compounds 110 and 225 were tested for cytotoxicity in human hepatocellular carcinoma cells (HepG2). The biofilm inhibitors were tested in triplicate at concentrations ranging from 30 to 60 μM. The approximate, gross cytotoxicity was measured using fluorometric detection of mitochondrial activity according to Nociari MM, et al. (1998) J. Immunol. Met. 213:157. Chlorpromazine (or its equivalent) was used as a positive control.

As shown in Table 10 below, compounds 110 and 225 exhibited considerable specificity for intended biofilm generating targets and did not significantly affect HepG2 cells.

TABLE 10

| | Inhibition at 30 μM - |
|---|---|
| Compound | Hep G2 cells |
| 110 | 2% |
| 225 | 1% |

### Example 5

### Dose/Response Evaluation of the Biofilm Inhibitors

In this example, dose/response characteristics of the biofilm inhibitors were measured using compound 108. The assay employed to measure such dose/response characteristics was adapted from a reported protocol. See Pratt and Kolter, 1998, Molecular Microbiology, 30: 285-293; and Li et al., 2001, J. Bacteriol., 183: 897-908.

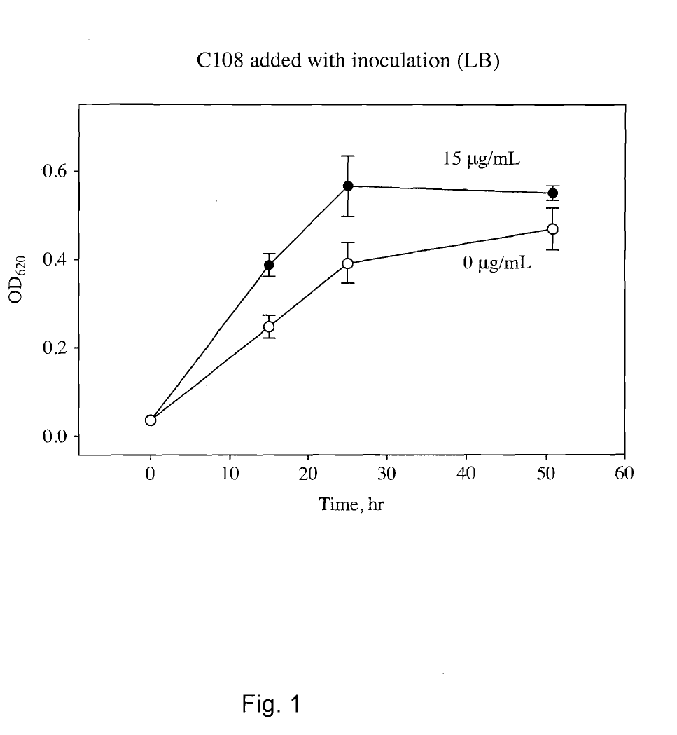

The dose/response behavior of compound 108 was tested against E. coli in LB media with and without 0.2% glucose. First, the effect of compound 108 on the viability of E. coli was measured. Compound 108 was added to a 96-well microtiter plate in triplicate wells to final concentrations of 0, 1, 2, 5, 10, and 15 μg/mL. Compound 108 was added to each well on the plate along with 50 μL of E. coli inoculum and 150 μL of LB and incubated at 37° C. for two days without shaking. After incubation, the plate was analyzed spectrophotometrically at 620 nm to quantitate the approximate levels of E. coli growth. The absorbance values observed for the test wells were averaged and compared to a negative control, i.e., wells containing no biofilm inhibitor. As shown in FIG. 1, compound 108 did not inhibit the growth of E. coli from 1 to 15 μg/mL.

The biofilm inhibitory effect of compound 108 was then evaluated at different times and concentrations. In the first assay, compound 108 was added to the test system at the same time as the E. coli inoculum. Specifically, compound 108 was added to the system at final concentrations of 0, 1, 2, 5, and 10 μg/mL along with 50 μL of E. coli inoculum and 150 μL of LB media to measure its ability to inhibit the formation of biofilms. In a second assay, compound 108 was added after a biofilm had formed, i.e., 24 hours after the assay plate was inoculated with E. coli in LB media. In a third assay, compound 108 was tested at 0, 1, 2, 5, 10, and 15 μg/mL against E. coli in LB media with 0.2% glucose (whereby compound 108 was introduced into the system at the time of inoculation).

The suspension culture in each of the assay systems described above was drained from the plate and the residual biofilm, if any, was washed three (3) times with water. A solution containing 0.1% crystal violet was added to each well and maintained thereon for approximately 20 minutes. Following the staining procedure, each well was rinsed three (3) times with water. Each well was then analyzed spectrophotometrically in a microtiter plate reader at 540 nm to quantitate approximate biofilm mass and inhibition of biofilm growth by comparison to a negative control, which included inoculum and growth media alone.

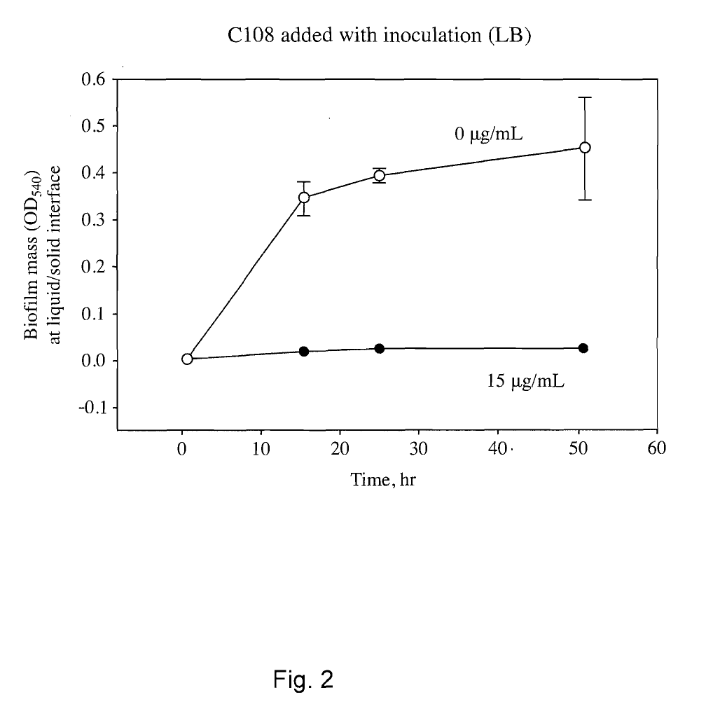

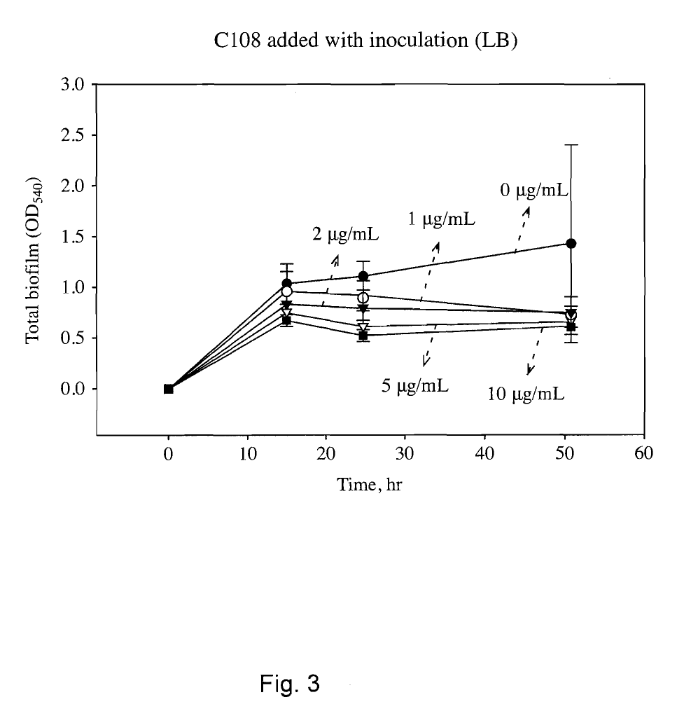

FIGS. 2 and 3 illustrate the dose/response behavior of compound 108 against E. coli in LB when added to the system along with the inoculum. These data show that compound 108 is effective at concentrations as low as 1 μg/mL in preventing biofilm formation—compared to the growth curve exhibited for the negative control (0 μg/mL). In addition, referring to FIG. 2, compound 108 at 15 μg/mL was shown to almost completely prevent biofilm growth from 0 through 50 hours of incubation.

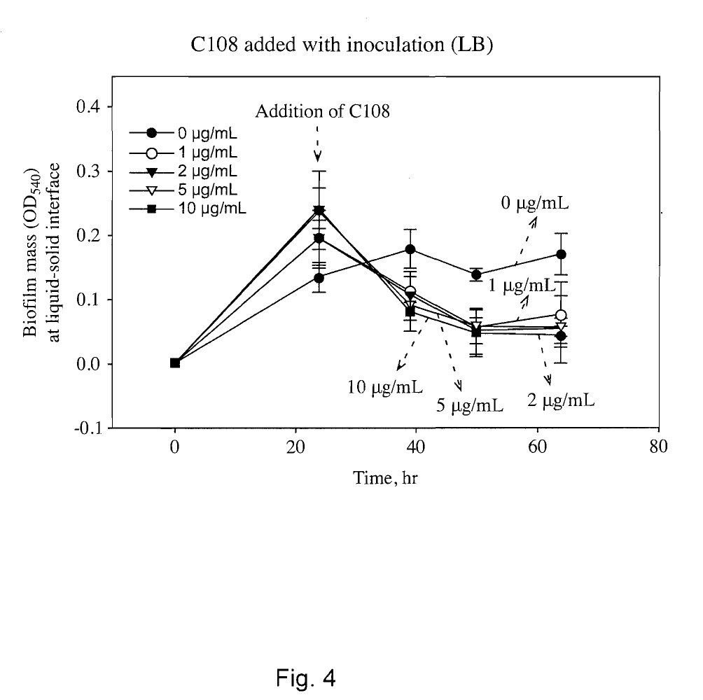

FIG. 4 shows the dose/response behavior of compound 108 against biofilm produced by E. coli in LB when added 24 hours after inoculation. These data show that compound 108 may be used at concentrations as low as 1 μg/mL to reduce existing biofilm mass. Referring to FIG. 4, the significant drop in biofilm mass is apparent upon addition of compound 108 at each of the concentrations tested. In particular; with only 24 hours of incubation with compound 108 (at each of the concentrations tested), i.e., at approximately 50 hours post inoculation, the estimated mass of existing biofilm decreased significantly.

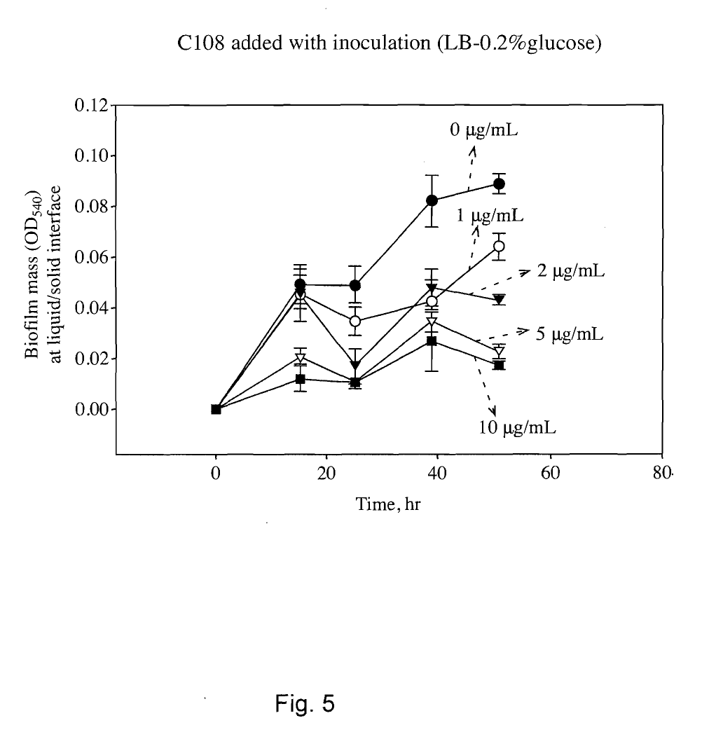

FIG. 5 shows the dose/response behavior of compound 108 against biofilm produced by E. coli in LB (with 0.2% glucose) when added to the system along with the inoculum. These data show that compound 108 is effective at reducing biofilm mass at concentrations as low as 1 μg/mL, and, more preferably, at 2 μg/mL or more in preventing biofilm formation by E. coli in LB with 0.2% glucose—compared to the growth curve exhibited for the negative control (0 μg/mL).

### Example 6

### Isolation and Purification of Compounds 116 and 188

The following provides a non-limiting example of a process by which Compounds 116 and 188 may be obtained and purified. First, an extraction step was carried out by grinding dried plant material derived from Diospyros dendo (Gabon, Africa) to a homogenous powder and sonicating the powder in an organic solvent, EtOH:EtOAc (50:50), and shaking the resulting mixture vigorously for exhaustive extraction. Next, flash chromatographic separation was carried out by adsorbing the extract solution onto silica powder and bringing the dried powder onto a silica column and eluting on a flash chromatography system using a step gradient of (1) 75% hexanes, 25% EtOAc, (2) 50% hexanes, 50% EtOAc, (3) 100% EtOAc, (4) 75% EtOAc, 25% MeOH, and (5) 50% EtOAc, 50% MeOH. Compounds 116 and 188 were located in the 100% EtOAc fraction. The fractions containing Compounds 116 and 188 were dried through rotary evaporation.

Following the flash chromatography isolation, preparative HPLC separation was carried out. The flash fraction material was dissolved into 100% MeOH. Using a parallel four-channel preparative HPLC system, the flash fractions were further separated into several individual fractions. Specifically, Compounds 188 and 116 were subjected to preparative HPLC C₁₈ chromatography using a 30% to 70% acetonitrile in water linear gradient over forty (40) minutes, collecting one (1) minute fractions. Compounds 188 and 116 resided in preparative HPLC fractions 33 and 38, respectively.

Following the isolation and purification of Compounds 188 and 116, the molecular weight and elemental formulas for such compounds were confirmed using positive-mode high resolution electron spray ionization mass spectrometry (HRESIMS). For Compound 188 positive-mode HRESIMS showed a [M+Na]⁺ ion peak at m/z 641.3816 (C₃₉H₅₄O₆Na requires 641.3818). ¹H and correlation spectrometry (COSY) NMR (CD₃OD) of Compound 188 showed the presence of a cis-p-coumaroyl moiety [δ7.67 (2H, d, J=8.4 Hz), 6.90 (1H, d, J=12.9 Hz), 6.76 (2H, d, J=8.4 Hz), 5.87 (1H, d, J=12.9 Hz)]. The ¹H NMR spectrum of Compound 188 was very similar to the ¹H NMR spectrum recorded for ursolic acid (Compound 110). However, the proton signal of H-3 in Compound 188 was shifted to a lower field at δ 4.62 (1H, m, H-3α), suggesting the O-cis-p-coumaroyl moiety to be at C-3. According to the molecular formula and the observed ¹H NMR spectrum, Compound 188 was determined to have a quaternary hydroxyl group at the C-20 position due to the fact that its ¹H NMR spectrum showed only one methyl doublet, instead of two doublets as in ursolic acid (Compound 110), and a one-proton doublet assigned to H-18 at δ 2.24 (1H, d, J=11.2 Hz, H-18β). The above was further supported by an isolated proton spin system of H-18, H-19 [δ 1.41.(1H, dt, J=11.2, 6.3 Hz, H-19α] and H-29 [δ 0.93 (3H, d, J=6.3 Hz)] in the COSY spectrum. Based on its biogenetic pathways and spectral information referenced above, the structure of Compound 188 was deduced as 3β-O-cis-p-coumaro yl-20β-hydroxy-12-ursen-28-oic acid.

Electrospray-ionization tandem mass spectrometry (ESIMS) analysis of Compound 116 showed a [M−H]⁻ ion peak at m/z 647, suggesting the chemical formula of C₄₀H₅₆O₇. The ¹H and COSY NMR (CD₃OD) spectra of Compound 116 showed the presence of a trans-feruloyl moiety [δ 7.07 (1H, dd, J=8.1, 1.8 Hz, H-2′), 6.81 (1H, d, J=8.1 Hz, H-3′), 7.20 (1H, d, J=1.8Hz), 7.62 (1H, d, J=15.8Hz, H-7′), 6.43 (1H, d, J=15.8 Hz), 3.90 (3H, s)]. Similar to Compound 188, the chemical shift recorded for H-3 suggested the feruloyl substituent to be at the C-3 position. Furthermore, the COSY spectrum showed the presence of another hydroxyl at C-2, based on a cross peak between H-3 at δ 5.02 (1H, d, J=2.2 Hz, H-3β) and a proton at δ 4.12 (1H, m, H-2β. This compound was therefore determined to be 3-O-trans-feruloyl-2-hydroxy-12-ursen-28-oic acid. Comparison of the ¹H NMR spectrum with the data reported for a (3β, 2α) derivative suggested Compound 116 has a different stereochemistry at C-3. This hypothesis was supported by nuclear overhauser effect spectroscopy (NOESY) and Compound 116 was therefore deduced as 3-O-trans-feruloyl-2α-hydroxy-12-ursen-28-oic acid.

Of course, those of ordinary skill in the art will appreciate that other known procedures may be employed to isolate and purify Compounds 188 and 116 (as well as the other biofilm-inhibiting compounds described herein)—which may depend on the nature of the plant extracts, extraction procedures employed, desired compound, and others.

The inventions being thus described, it will be obvious that the same may be varied in many ways. Such variations are not to be regarded as a departure from the spirit and scope of the inventions and all such modifications are intended to be included within the scope of the following claims.

What is claimed:

1. A compound selected from the group consisting of: 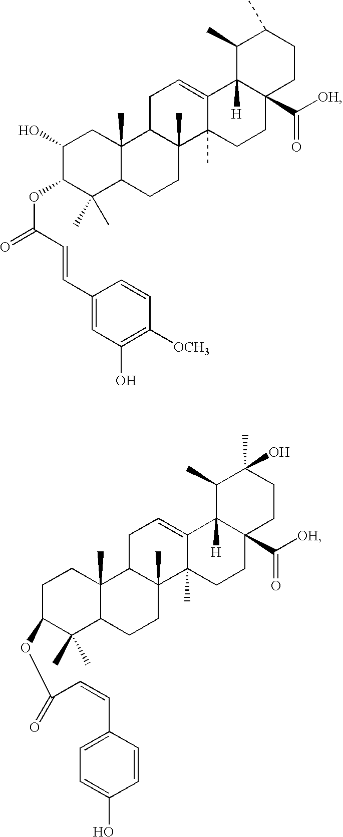
and salts, hydrates, solvates, and N-oxides of the foregoing.

2. A compound as set forth in claim 1 wherein the compound is selected from the group consisting of: 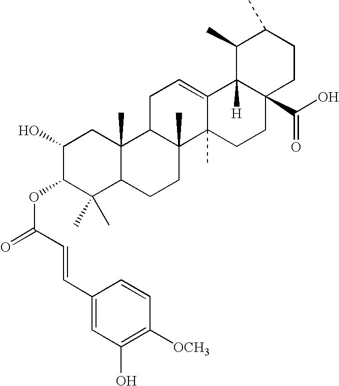
and salts, hydrates, solvates, and N-oxides thereof.

3. A compound as set forth in claim 1, wherein the compound is selected from the group consisting of: 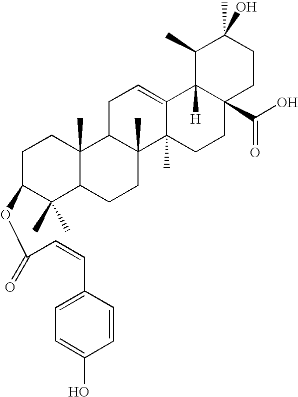
and salts, hydrates, solvates, and N-oxides thereof.

4. A compound as set forth in claim 1 wherein the compound has the chemical structure:

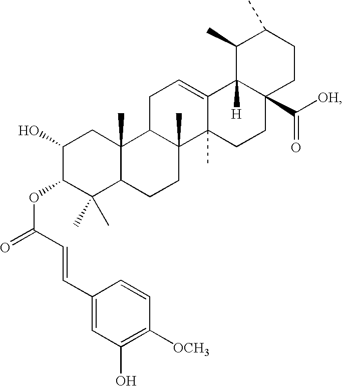

5. A compound as set forth in claim 1 wherein the compound has the chemical structure:

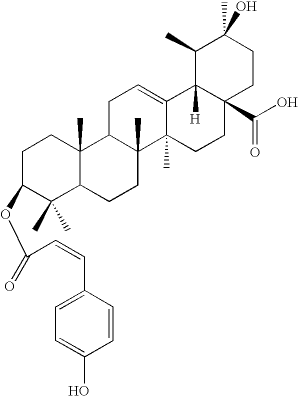

6. A composition for inhibiting or treating a biofilm comprising an effective amount of a compound selected from the group consisting of 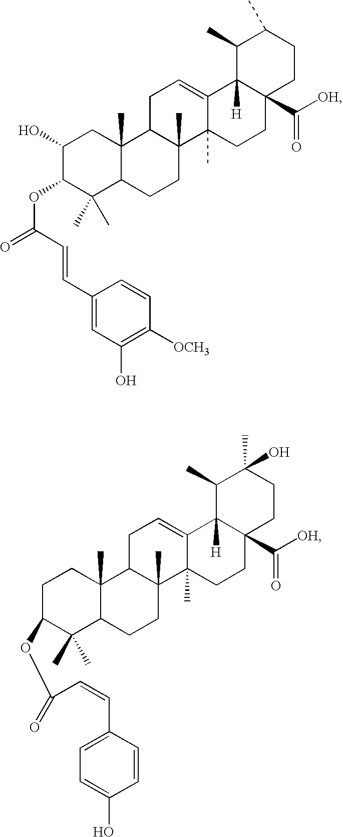
and salts, hydrates, solvates, and N-oxides of the foregoing.

7. A composition as set forth in claim 6 wherein the compound is selected from the group consisting of compounds having the chemical structure: 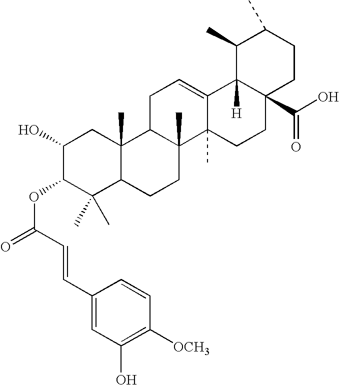
and salts, hydrates, solvates, and N-oxides of the foregoing.

8. A composition as set forth in claim 6 wherein the compound is selected from the group consisting of compounds having the chemical structure: 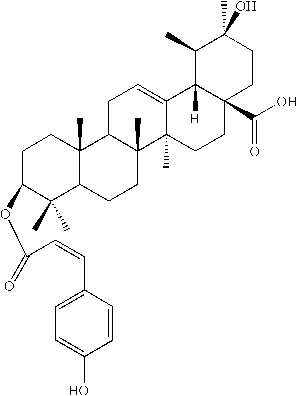
and salts, hydrates, solvates, and N-oxides thereof.

9. A composition as set forth in claim 6 wherein the compound has the chemical structure:

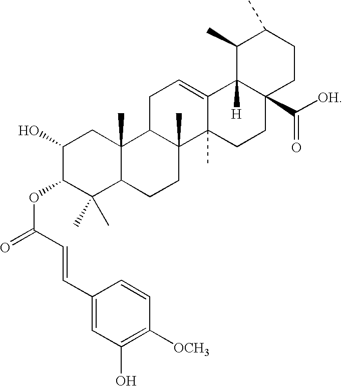

10. A composition as set forth in claim 6 wherein the compound has the chemical structure: [chemical structure]
